(19) United States
(12) Patent Application Publication
(10) Pub. No.: US 2021/0291176 A1
(43) Pub. Date: Sep. 23, 2021

(54) RAPID DIAGNOSTIC TEST WITH BLISTER PACK

(71) Applicant: Detect, Inc., Guilford, CT (US)

(72) Inventors: Todd Roswech, Ivoryton, CT (US); John H. Leamon, Stonington, CT (US); Jonathan M. Rothberg, Guilford, CT (US)

(73) Assignee: Detect, Inc., Guilford, CT (US)

(21) Appl. No.: 17/203,504

(22) Filed: Mar. 16, 2021

Related U.S. Application Data
(60) Provisional application No. 63/081,201, filed on Sep. 21, 2020.
(60) Provisional application No. 63/068,303, filed on Aug. 20, 2020.
(60) Provisional application No. 63/065,131, filed on Aug. 13, 2020.
(60) Provisional application No. 63/059,928, filed on Jul. 31, 2020.
(60) Provisional application No. 63/053,534, filed on Jul. 17, 2020.
(60) Provisional application No. 63/036,887, filed on Jun. 9, 2020.
(60) Provisional application No. 63/027,859, filed on May 20, 2020.
(60) Provisional application No. 63/027,874, filed on May 20, 2020.
(60) Provisional application No. 63/027,890, filed on May 20, 2020.
(60) Provisional application No. 63/027,864, filed on May 20, 2020.
(60) Provisional application No. 63/027,878, filed on May 20, 2020.
(60) Provisional application No. 63/027,886, filed on May 20, 2020.
(60) Provisional application No. 63/022,534, filed on May 10, 2020.
(60) Provisional application No. 63/022,533, filed on May 10, 2020.
(60) Provisional application No. 63/013,450, filed on Apr. 21, 2020.
(60) Provisional application No. 63/010,578, filed on Apr. 15, 2020.
(60) Provisional application No. 63/010,626, filed on Apr. 15, 2020.
(60) Provisional application No. 63/002,209, filed on Mar. 30, 2020.
(60) Provisional application No. 62/991,039, filed on Mar. 17, 2020.

(51) Int. Cl. B01L 3/00

(52) U.S. Cl. CPC B01L 3/502715; B01L 3/527; B01L 2400/0644; B01L 2200/0689; B01L 2300/0825; B01L 2200/16

(57) ABSTRACT

Provided herein, in some embodiments, are rapid diagnostic tests to detect one or more target nucleic acid sequences (e.g., a nucleic acid sequence of one or more pathogens). In some embodiments, the pathogens are viral, bacterial, fungal, parasitic, or protozoan pathogens, such as SARS-CoV-2 or an influenza virus. Further embodiments provide methods of detecting genetic abnormalities. Diagnostic tests comprising a sample-collecting component, one or more reagents (e.g., lysis reagents, nucleic acid amplification reagents), and a detection component (e.g., a component comprising a lateral flow assay strip) are provided.

### RELATED APPLICATIONS

This application claims the benefit under 35 U.S.C. § 119(e) of U.S. Provisional Application Ser. No. 62/991,039, filed Mar. 17, 2020, U.S. Provisional Application Ser. No. 63/002,209, filed Mar. 30, 2020, U.S. Provisional Application Ser. No. 63/010,578, filed Apr. 15, 2020, U.S. Provisional Application Ser. No. 63/010,626, filed Apr. 15, 2020, U.S. Provisional Application Ser. No. 63/013,450, filed Apr. 21, 2020, U.S. Provisional Application Ser. No. 63/022,534, filed May 10, 2020, U.S. Provisional Application Ser. No. 63/022,533, filed May 10, 2020, U.S. Provisional Application Ser. No. 63/036,887, filed Jun. 9, 2020, U.S. Provisional Application Ser. No. 63/081,201, filed Sep. 21, 2020, U.S. Provisional Application Ser. No. 63/065,131, filed Aug. 13, 2020, U.S. Provisional Application Ser. No. 63/059,928, filed Jul. 31, 2020, U.S. Provisional Application Ser. No. 63/068,303, filed Aug. 20, 2020, U.S. Provisional Application Ser. No. 63/027,859, filed May 20, 2020, U.S. Provisional Application Ser. No. 63/027,874, filed May 20, 2020, U.S. Provisional Application Ser. No. 63/027,890, filed May 20, 2020, U.S. Provisional Application Ser. No. 63/027,864, filed May 20, 2020, U.S. Provisional Application Ser. No. 63/027,878, filed May 20, 2020, U.S. Provisional Application Ser. No. 63/027,886, filed May 20, 2020, and U.S. Provisional Application Ser. No. 63/053,534, filed Jul. 17, 2020, each of which is hereby incorporated by reference in its entirety.

### FIELD

The present invention generally relates to diagnostic devices, systems, and methods for detecting the presence of a target nucleic acid sequence.

### BACKGROUND

The ability to rapidly diagnose diseases—particularly highly infectious diseases—is critical to preserving human health. As one example, the high level of contagiousness, the high mortality rate, and the lack of a treatment or vaccine for the coronavirus disease 2019 (COVID-19) have resulted in a pandemic that has already infected millions and killed hundreds of thousands of people. The existence of rapid, accurate COVID-19 diagnostic tests could allow infected individuals to be quickly identified and isolated, which could assist with containment of the disease. In the absence of such diagnostic tests, COVID-19 may continue to spread unchecked throughout communities.

### SUMMARY

In some embodiments, a diagnostic test includes a housing, a first blister chamber formed in the housing containing a first reagent and a lateral flow assay strip disposed in the housing. In some embodiments, the diagnostic test also includes a fluidic channel between the blister chamber and the lateral flow assay strip, and a seal positioned between the first blister chamber and the lateral flow assay strip. Applying a threshold force to the first blister chamber is configured to open the seal to fluidly connect the first blister chamber to the fluidic channel.

In some embodiments, a method of performing a diagnostic test includes depositing a sample into a first blister chamber through a sample port formed in a housing, allowing the sample to react with a first reagent in the first blister chamber to form a first solution, applying a threshold force to a first blister chamber to break a first frangible seal holding the first solution in the first blister chamber, and allowing the first solution to flow toward a lateral flow assay strip disposed in the housing.

In some embodiments, a method of making a diagnostic test includes placing a first reagent in a first blister chamber, placing a second reagent in a second blister chamber, positioning a first seal between the first blister chamber and the second blister chamber, and placing a lateral flow assay strip in a third chamber. In some embodiments, the method also includes positioning a second seal between the second blister chamber and the lateral flow assay strip.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 shows, according to some embodiments, a diagnostic device comprising a plurality of blister packs;

FIGS. 2A-2C show blister packs, according to some embodiments;

FIGS. 3A-3D show a process of using one embodiment of a blister pack to perform a diagnostic test;

FIG. 4 is a flow chart for one embodiment of making a diagnostic test;

FIG. 5 is a flow chart for one embodiment of performing a diagnostic test;

FIG. 6 is depicts one embodiment of a diagnostic test performing a diagnostic testing process; and

FIG. 7 depicts a schematic of one embodiment of a lateral flow assay strip readout.

### DETAILED DESCRIPTION

Conventional nucleic acid tests for various diseases requires trained medical professional to collect samples and process those samples in a sterile environment in a laboratory. Such a process is time consuming, resulting in a delay in providing results to patients. Additionally, such tests require a patient to visit a location where a sample may be collected and transported in a sterile manner to an appropriate processing location. Travel to and from locations may risk spread of the disease being tested for and may inadvertently expose medical personnel to the disease.

As the COVID-19 pandemic has highlighted, there is a critical need for rapid, accurate systems and methods for diagnosing diseases—particularly infectious diseases. In the absence of diagnostic testing, asymptomatic infected individuals may unknowingly spread the disease to others, and symptomatic infected individuals may not receive appropriate treatment. With testing, however, infected individuals may take appropriate precautions (e.g., self-quarantine) to reduce the risk of infecting others and may receive targeted treatment as helpful.

While diagnostic tests for various diseases, including COVID-19, are known, such tests often require specialized knowledge of laboratory techniques and/or expensive laboratory equipment. For example, polymerase chain reaction (PCR) tests generally require skilled technicians and expensive, bulky thermocyclers. In addition, there is a need for diagnostic tests that are both rapid and highly accurate. Known diagnostic tests with high levels of accuracy often take hours, or even days, to return results, and more rapid tests generally have low levels of accuracy. Many rapid diagnostic tests detect antibodies, which generally can only reveal whether a person has previously had a disease, not whether the person has an active infection. In contrast, nucleic acid tests (i.e., tests that detect one or more target nucleic acid sequences) may indicate that a person has an active infection.

In view of the above, the inventors have recognized the benefits of a rapid diagnostic test that is usable by user who may not be medical professionals. In particular, the inventors have recognized the benefits of a rapid diagnostic tests employing fluid reservoirs having blister chambers and seals that may be easily punctured to fluidly connect various elements of the rapid diagnostic test in sequence while maintaining sterility. Such a rapid diagnostic test including blister chambers may allow users to perform tests and receive results in a rapid manner without necessarily requiring input from trained medical staff. Telemedicine, or applications may be employed to further enhance the usability of the rapid diagnostic test, such that a variety of diseases such as COVID-19, influenza, (or any target nucleic acid) may be tested for in an at-home or point-of-care environment.

The present disclosure provides diagnostic devices, systems, and methods for rapidly detecting one or more target nucleic acid sequences (e.g., a nucleic acid sequence of a pathogen, such as SARS-CoV-2 or an influenza virus). A diagnostic system, as described herein, may be self-administrable and comprise a sample-collecting component (e.g., a swab) and a diagnostic device. The diagnostic device may comprise a blister pack detection device, according to some embodiments. In some cases, the diagnostic device comprises a detection component (e.g., a lateral flow assay strip), results of which are self-readable, or automatically read by a computer algorithm. In certain embodiments, the diagnostic device further comprises one or more reagents (e.g., lysis reagents, nucleic acid amplification reagents, CRISPR/Cas detection reagents). In certain other embodiments, the diagnostic system separately includes one or more reaction tubes comprising the one or more reagents. The diagnostic device may also comprise an integrated heater, or the diagnostic system may comprise a separate heater. The isothermal amplification technique employed yields not only fast but very accurate results.

Provided herein are a number of diagnostic tests useful for detecting target nucleic acid sequences. According to exemplary embodiments described herein, it may be desirable to selectively move solutions contained in different chambers of a diagnostic test. In particular, the inventors have recognized that moving reagents through a diagnostic test at specific times in a sterile manner may provide rapid, accurate results. Accordingly, the inventors have recognized the benefits of employing in a diagnostic test a blister pack including one or more blister chambers. The diagnostic tests including one or more blister chambers, as described herein, are able to be performed in a point-of-care (POC) setting or home setting without specialized equipment. In some aspects, a cartridge or housing includes one or more blister chambers that enable reliable, sterile transmission of one or more solutions throughout a testing process. The blister chambers may allow a user to apply a threshold force to an exterior of a blister chamber to break a frangible seal and/or transfer the contents of the blister chamber to another portion of the diagnostic test. As the force may be applied externally and no tools may be required to transfer solutions through the diagnostic test, the internal sterility of the diagnostic test may be maintained throughout a testing process. Multiple blister chambers may be arranged in sequence so that the steps of performing a diagnostic test, including fluid transfers to a lateral flow assay strip, are simple for an at-home user.

### Blister Pack

In some embodiments of the present technology, a blister pack is described, which blister pack may be used as part of a diagnostic test. In some embodiments, a blister pack may comprise one or more chambers, in which each chamber may be a “blister” of the blister pack. In some cases, each chamber may comprise one or more reagents (e.g., lysis reagents, nucleic acid amplification reagents, and the like) and/or one or more buffers (e.g., a dilution buffer). In certain, a chamber may be separated from an adjacent chamber by a breakable seal (e.g., a frangible seal) or a valve (e.g., a rotary valve). The blister pack described may be used in any diagnostic test with which it can be advantageous, including the exemplary test described herein.

Diagnostic devices and systems described herein may comprise any number of blister packs, arranged in such a way so as to process a sample as described herein. In some embodiments, the blister packs may comprise one or more seals (e.g., differential seals, frangible seals) that allow reagents to be delivered in a controlled manner (e.g., using differential seal technology). In some embodiments, a frangible seal may be formed of a metal foil, elastomeric film, flexible plastic, or any other suitable breakable material. In some embodiments, the blister packs may comprise one or more chambers, where each chamber may comprise one or more reagents. In certain embodiments, one or more chambers may store one or more reagents in solid form (e.g., lyophilized, dried, crystallized, air jetted, etc.), and one or more chambers may store one or more reagents and/or buffers in liquid form. In some cases, a chamber comprising one or more reagents in solid form may be separated from a chamber comprising one or more reagents and/or buffers in liquid form by a seal (e.g., a frangible seal). In some cases, breaking the frangible seal may result in the one or more solid reagents being suspended in the one or more liquid reagents and/or buffers. In some cases, the suspended solid reagent(s) may be added to a sample.

In some embodiments of the present technology, the delivery of each reagent in a blister pack may be fully automated. For example, the user may insert a sample in a sample collection region of the blister pack and then activate the blister pack. Upon activation, all of the reagents may be added to the sample in the correct amount and at the appropriate time, such that the sample is processed as described herein. In some embodiments, the blister pack may further comprise a detection component (e.g., a lateral flow assay strip). The detection component may alert the user as to whether the sample was positive or negative for the target nucleic acid sequence.

In some embodiments, a diagnostic test includes a housing having a first blister chamber containing a first reagent. The diagnostic test also includes a lateral flow assay strip disposed in the housing. A fluidic channel selectively connects the blister chamber and the lateral flow assay strip, such that a solution may flow from the blister chamber to the lateral flow assay strip. A seal positioned between the blister chamber and the lateral flow assay strip (e.g., in the fluidic channel) is configured to prevent the solution from flowing to the lateral flow assay strip until the seal is opened. The blister chamber may be configured to receive an external force, where when the external force reaches a threshold force the seal opens and allows a solution inside of the first blister chamber to flow toward the lateral flow assay strip. In some embodiments, the diagnostic test may include additional blister chambers positioned between the first blister chamber and the lateral flow assay strip. In such an embodiment, application of the threshold force to the first blister chamber may transfer a solution contained therein to an adjacent blister chamber between the first blister chamber and the lateral flow assay strip. Accordingly, a threshold force may then be applied to the next blister chamber and so on until a final solution is transferred to the lateral flow assay strip. Of course, in some embodiments, one or more blister chambers may not be sequentially connected. For example, in one embodiment a plurality of blister chambers may be connected to an amplification blister chamber, where solutions from the plurality of blister chambers are pooled in the amplification blister chamber. Of course, combinations of the above arrangements are also contemplated, where some blister chambers are arranged in sequence and pool with at least one non-sequential chamber in a separate central chamber.

According to exemplary embodiments described herein, multiple blister chambers may be employed in a diagnostic test. In some embodiments, a diagnostic test may include two blister chambers. In other embodiments, a diagnostic test may include three blister chambers, four blister chambers, or five blister chambers. A diagnostic test may also include any suitable number of fluid chambers that are not arranged as blisters or are otherwise not configured to receive an external force. Non-blister chambers and blister chambers may be combined in any suitable number and arrangement in a diagnostic test. In some embodiments, a lateral flow assay strip of a diagnostic test may be disposed in a blister chamber. In other embodiments, a lateral flow assay strip may be disposed in a non-blister chamber.

According to exemplary embodiments described herein, a blister chamber of a diagnostic test may include a sample port configured to receive a sample from a patient. The sample port may be configured to receive a sample from various testing arrangements. For example, the sample port may be configured to receive a swab. In some embodiments, the sample port may be configured as a septum configured to open when force is applied to the septum with the sample. Accordingly, the sample may be taken from a patient and then easily inserted into the blister chamber through the septum. In other embodiments, a blister chamber may include a removable cap which is removed to allow a sample to be deposited in the blister chamber. In some embodiments, the sample port may include a frangible seal that is broken by the sample or another puncturing tool. Of course, any suitable cap or seal may be employed to form a sample port through which a sample may be deposited in a blister chamber, as the present disclosure is not so limited.

According to exemplary embodiments described herein, a blister chamber may include a reagent forming a component of a diagnostic test. A reagent may be a liquid solution or may be in solid form. For example, in some embodiments a reagent may be buffer solution or an amplification solution. In some embodiments, a reagent may be a lyophilized solid, where the solid is configured to dissolve in a solution contained in a connected blister chamber. The reagent may be a solid or liquid amplification reagent, buffer reagent, lysis reagent, or another desired reagent.

In some embodiments, a blister chamber may be configured to receive an external force to open a seal separating the blister chamber from a fluidic channel or lateral flow assay strip. Depending on the particular reagent and/or feeling for the end user, a blister chamber may be formed of a material that provides feedback to the user in combination with a seal. In some embodiments, the blister chamber may be formed of a rigid film material (e.g., metal foil), which provides a rigid feeling when applying force to the blister chamber. In other embodiments, the blister chamber may be formed of a flexible film material (e.g., an elastomeric or flexible plastic material). Of course, a blister chamber may be formed of any suitable material, as the present disclosure is not so limited.

According to exemplary embodiments described herein, a method of performing a diagnostic test includes depositing a sample into a blister chamber through a sample port formed in a housing. In some embodiments, depositing the sample in the blister chamber may include pushing the sample through a septum forming the sample port. In the method may also include allowing the sample the react with a first reagent in the blister chamber to form a first solution. The first reagent may be a sample buffer solution. The method may also include applying a threshold force to an external portion of the blister chamber to break a frangible seal holding the first solution inside the blister chamber. Once the seal is broken, the solution may be forced and/or allowed to flow toward a lateral flow assay strip disposed in the housing. In some embodiments, once the seal of the first blister chamber is opened, the first solution may flow into a second blister chamber. The second blister chamber may include a second reagent configured to react with the first solution. Like the first blister chamber, the second blister chamber may be configured to receive an external force. When a second threshold force is applied to the second blister chamber, a second seal may be opened to allow the solution contained inside of the second blister back. In embodiments where more than two blister packs are employed, the process of applying a threshold force to the additional blister chambers may be repeated to sequentially release solutions and/or solid reagents.

In some embodiments, a method of making a diagnostic test includes placing a first reagent in a first blister chamber and placing a second reagent in the second blister chamber. In some embodiments, the first and second reagents may be liquid solutions, lyophilized solids. In some cases, one of the first blister chamber and second blister chamber may include a liquid solution, and the other may include a lyophilized solid. In some embodiments, the liquid solution in one of the blister chambers may be configured to hydrate a lyophilized solid in another blister chamber. The method may also include positioning a first seal between the first blister chamber and the second blister chamber. When the first seal is opened (e.g., by application of an external threshold force the first blister chamber and/or second blister chamber) the first blister chamber may be fluidly connected to the second blister chamber. The method may also include placing a lateral flow assay strip in a third chamber and placing a second seal between the second blister chamber and the lateral flow assay strip Like the first seal, opening the second seal may fluidly connect the second blister chamber to the lateral flow assay strip.

It should be noted that while in exemplary embodiments described herein a threshold force is externally applied to a blister chamber to open a seal, in other embodiments a puncturing tool or external force may instead be applied to the seal itself to open the seal. In this regard, a diagnostic test is not limited to external force being applied to the blister chamber to open the seal, and any suitable opening arrangement may be employed.

In some embodiments, a diagnostic device comprises one or more blister packs. In some embodiments, a blister pack comprises one or more chambers. In some cases, each chamber may comprise one or more reagents (e.g., lysis reagents, nucleic acid amplification reagents) and/or one or more buffers (e.g., dilution buffer). In certain, a chamber may be separated from an adjacent chamber by a breakable seal (e.g., a frangible seal) or a valve (e.g., a rotary valve).

Diagnostic devices and systems described herein may comprise any number of blister packs, arranged in such a way so as to process a sample as described herein. In some embodiments, the blister packs comprise one or more seals (e.g., differential seals, frangible seals) that allow reagents to be delivered in a controlled manner (e.g., using differential seal technology) or an uncontrolled manner (e.g., using a burstable, frangible seal). In some embodiments, the blister packs comprise one or more chambers, where each chamber comprises one or more reagents. In certain embodiments, one or more chambers store one or more reagents in solid form (e.g., lyophilized, dried, crystallized, air jetted), and one or more chambers store one or more reagents and/or buffers in liquid form. In some cases, a chamber comprising one or more reagents in solid form may be separated from a chamber comprising one or more reagents and/or buffers in liquid form by a seal (e.g., a frangible seal). In some cases, breaking the frangible seal may result in the solid reagents being suspended in the one or more liquid reagents and/or buffers. In some cases, the suspended solid reagents may be added to a sample.

In some embodiments, the delivery of each reagent in a blister pack is fully automated. For example, the user may insert a sample in a sample collection region of the blister pack and then activate the blister pack. Upon activation, all of the reagents may be added to the sample in the correct amount and at the appropriate time, such that the sample is processed as described herein. In some embodiments, the blister pack further comprises a detection component (e.g., a lateral flow assay strip). The detection component may alert the user as to whether the sample was positive or negative for the target nucleic acid sequence.

Turning to the figures, specific non-limiting embodiments of diagnostic tests including one or more blister chambers are described in further detail. It should be understood that the various features and methods of the blister chambers described relative to these embodiments may be used either individually and/or in any desired combination, as the disclosure is not limited to only the specific embodiments described herein.

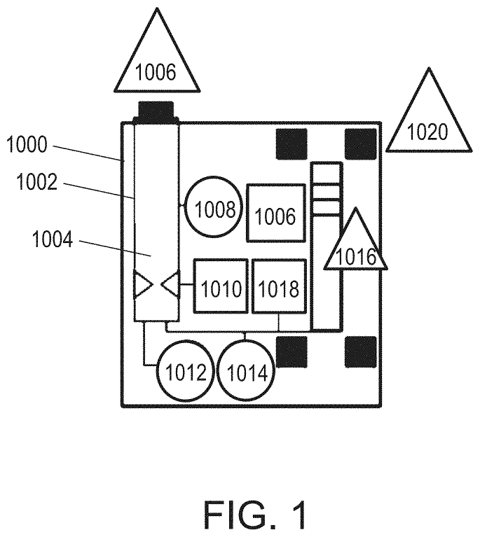

One embodiment is shown in FIG. 1. In FIG. 1, diagnostic device 1000 comprises tube 1002 containing reaction buffer 1004. In certain embodiments, diagnostic device 1000 comprises a heater in thermal communication with tube 1002.

In operation, a sample may be added through sample port 1006. A first blister pack 1008 comprising one or more lysis and/or decontamination reagents (e.g., UDG) are released from blister pack 1008 into tube 1002. In some embodiments, tube 1002 may be heated by a heater (not shown in FIG. 1). In some cases, mechanism 1010 provides a physical mechanism to reduce sample volume as needed. In certain embodiments, one or more amplification reagents are released from amplification blister pack 1012 into tube 1002. In some instances, a dilution buffer may optionally be released from dilution blister pack 1014 into tube 1002. The sample is then flowed across a lateral flow assay strip 1016, with mechanism 1018 ensuring that the sample accesses lateral flow assay strip 1016 at the appropriate time (e.g., after the processing is complete). In certain embodiments, one or more markers 1020 (e.g., one or more ArUco markers) are provided to facilitate image alignment and. In some embodiments, device 1000 comprises a QR barcode that may encode device information and may be used by a software-based application (e.g., to pair the user to the test result). In some embodiments, the blister packs may be frangible blister packs, wherein the lyophilized reagent and its buffer or solution are kept in two separate blister packs and then mixed together before interacting with the sample. In some embodiments, the two separate blister packs are adjacent to one another and the seal between them is broken to combine them.

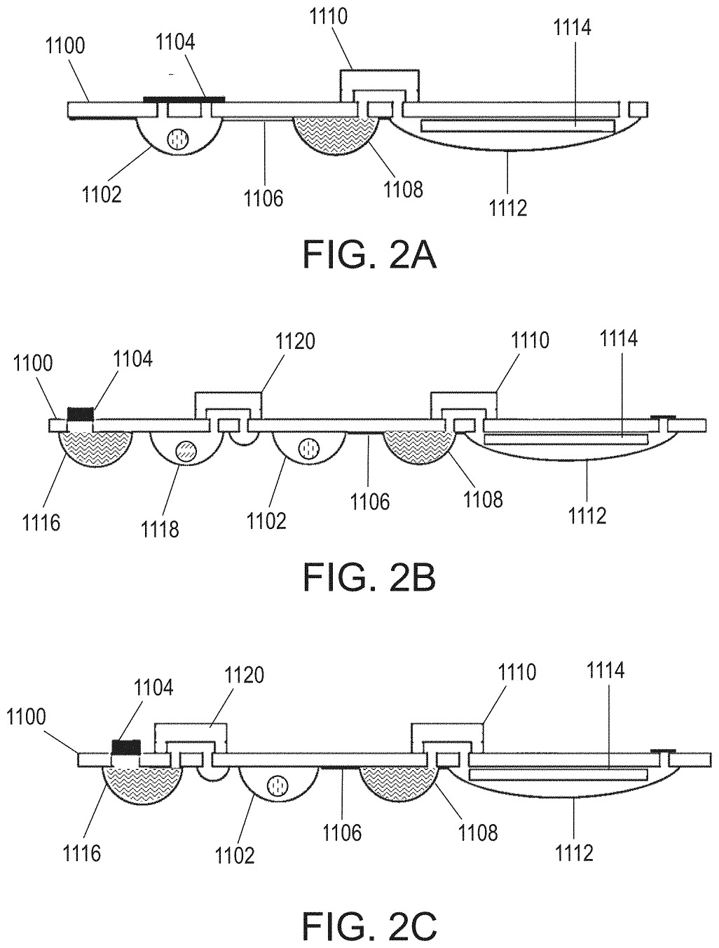

An exemplary blister pack is shown in FIG. 2A. In FIG. 2A, blister pack 1100 comprises first chamber 1102, sample port 1104, seal 1106, second chamber 1108, valve 1110, third chamber 1112, and lateral flow assay strip 1114. First chamber 1102 may comprise one or more amplification reagents (e.g., LAMP, RPA, NEAR reagents) in solid form (e.g., lyophilized). Second chamber 1108 may comprise a dilution buffer. Third chamber 1112 may comprise a lateral flow assay strip. First chamber 1102 and second chamber 1108 may be separated by a breakable seal (e.g., a frangible seal). Second chamber 1108 and third chamber 1112 may be separated by a valve (e.g., a rotary valve). Other blister pack configurations are possible.

FIG. 2B shows a blister pack embodiment comprising one or more chambers comprising one or more lysis reagents. In FIG. 2B, blister pack 1100 comprises not only first chamber 1102 comprising one or more amplification reagents, second chamber 1108 comprising a dilution buffer, and third chamber 1112 comprising lateral flow assay strip 1114, but also fourth chamber 1116 comprising a sample buffer and fifth chamber 1118 comprising one or more lysis reagents. In some embodiments, the one or more lysis reagents comprise one or more enzymes and/or detergents. In some embodiments, the one or more lysis reagents are in solid form (e.g., lyophilized). As shown in FIG. 2B, blister pack 1100 further comprises sample port 1104, which allows injection of a sample into fourth chamber 1116, and valve 1120 (e.g., a rotary valve). Furthermore, the blister pack 1100 includes a second valve 1120 configured to allow selective fluid communication between the fifth chamber 1118 and the first chamber 1102.

FIG. 2C shows a blister pack embodiment configured to facilitate thermal lysis and isothermal nucleic acid amplification. In FIG. 2C, blister pack 1100 comprises fourth chamber 1116 comprising a sample buffer, first chamber 1102 comprising one or more amplification reagents (e.g., LAMP, RPA, NEAR reagents), second chamber 1108 comprising a dilution buffer, and third chamber 1112 comprising lateral flow assay strip 1114. In some embodiments, fourth chamber 1116 may be heated by an external heater after addition of a sample to the chamber. Furthermore, the blister pack 1100 includes a second valve 1120 configured to allow selective fluid communication between the fourth chamber 1116 and the first chamber 1102.

In another embodiment, the sample is processed initially in a sample tube, and then injected into a sample port of the cartridge (blister pack), where it undergoes amplification (e.g., RPA, LAMP, NEAR, or other isothermal amplification process) and then is added to a lateral flow device to be analyzed. In a further embodiment, the swab is mixed with the sample buffer and a lyophilized lysis mix is added when a frangible seal is broken. The sample is then moved to a lyophilized amplification mix comprising the reagents necessary for RPA, LAMP, or other isothermal amplification techniques. Similarly, a dilution buffer is added to the lyophilized mixture when its frangible seal is broken. The sample, after processing, is then added to a lateral flow device to be analyzed. In some embodiments, the lysis is accomplished by enzymatic and/or detergent lysis mechanisms. In a further embodiment, heat lysis is used. That is, the sample is added to the sample buffer and then heat is applied to lyse the sample. After the sample has been lysed, it is then moved to a lyophilized amplification mix chamber (blister). Similarly, a dilution buffer is added to the lyophilized mixture when its frangible seal is broken. The sample, after processing, is then added to a lateral flow device to be analyzed. In some embodiments, each of the steps is separated by a rotary valve, which controls the flow of the sample into the next chamber (blister).

A further embodiment of the blister pack configuration comprises a swab in conjunction with a blister pack. A sample is taken using a swab. The swab is added to a tube comprising buffer and incubated for 10 minutes at room temperature. Then, a cap comprising one or more lysis reagents is added to the tube. Adding the cap dispenses the lysis reagents into the buffer and sample. The mixture is then heated at 95° C. for three minutes but the invention is not so limited. Other temperatures are envisioned. In some embodiments, the heating is accomplished with any heater described herein (e.g., boiling water, a fixed heat source). The reaction mixture is then allowed to cool for 1 minute, but this time period is not limiting as other time periods are envisioned. The resulting reaction mixture is then injected into a sample port of the blister pack (e.g., using a pipette). The blister pack is then sealed with seal tape and then shaken or otherwise agitated (e.g., shaken) for 10 seconds but this time period is not limiting. The blister pack is heated for 20 minutes but this time period also is not limiting. In some embodiments, the blister pack may be placed in a user's clothing pocket (e.g., back pocket of pants, front pocket of pants, front pocket of shirt) to heat the blister pack using the user's body heat. The user then pushes on a first blister to release a one or more amplification reagents (e.g., one or more reagents for LAMP, RPA, NEAR, or other isothermal amplification methods). The user presses on a second blister to release the dilution buffer and turns a valve to permit the mixture to proceed to a lateral flow strip after the appropriate amount of processing. The lateral flow strip may indicate whether one or more target nucleic acid sequences are present in the sample. In some embodiments, the results on the lateral flow strip are interpreted using a mobile software-based application, downloadable to a smart device, such as that described herein.

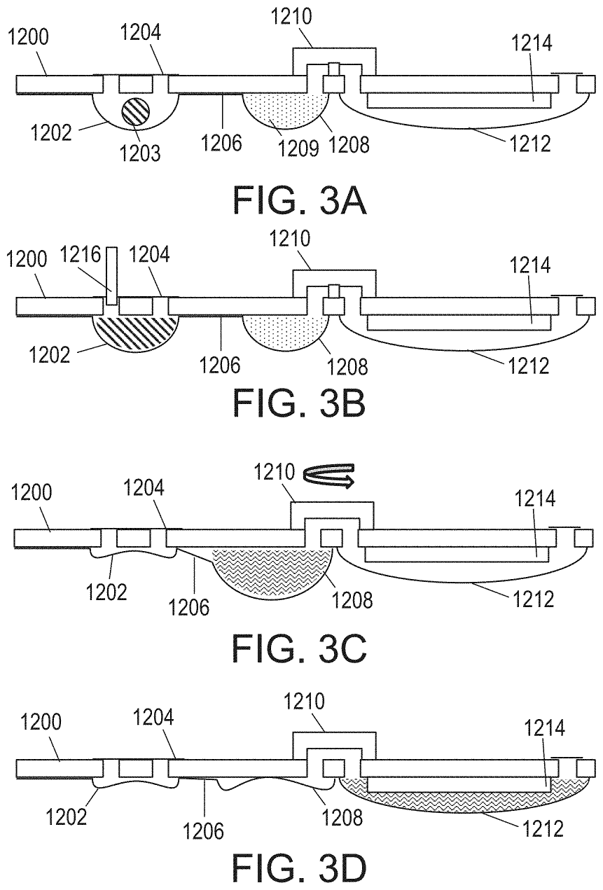

FIGS. 3A-3D depict a process of completing a diagnostic testing process using one embodiment of a blister pack diagnostic test. As shown in FIG. 3A, the blister pack 1200 comprises first chamber 1202, sample port 1204, seal 1206, second chamber 1208, valve 1210, third chamber 1212, and lateral flow assay strip 1214. According to the embodiment of FIGS. 3A-3D, the first chamber 1202 may comprise one or more amplification reagents 1203 (e.g., LAMP, RPA, NEAR reagents) in solid form (e.g., lyophilized) The second chamber 1108 comprises a dilution buffer 1209 which is a liquid solution. The third chamber 1212 houses the lateral flow assay strip 1214. As shown in FIG. 3A, the first chamber 1202 and second chamber 1208 may be separated by a breakable seal 1206 (e.g., a frangible seal). When a threshold force is applied to the first blister chamber 1202 and/or the second blister chamber 1208, the breakable seal may be configured to open (i.e., burst). As shown in FIG. 3A, the second chamber 1208 and third chamber 1212 are separated by a rotary valve, where the valve may be rotated to open or close a fluidic channel between the second blister chamber 1208 and the third chamber 1212. That is, rotating the rotary valve may switch the valve between an open state and a closed state.

FIG. 3A may represent a state in which the diagnostic test is delivered to an end user before the diagnostic testing process begins. As shown in FIG. 3B, the first step of performing a diagnostic test may include taking a sample, and then placing that sample in the first blister chamber 1202. In particular, as shown in FIG. 3B, placing the sample in the first blister chamber includes moving a pipette 1216 through the sample port 1204. According to the embodiment of FIGS. 3A-3D, the sample port may be a septum that is non-destructively opened by the pipette 1216. As shown in FIG. 3B, the pipette 1216 may be used to deposit a liquid sample into the first blister chamber 1202. The liquid sample may react with the solid amplificant reagents 1203 shown in FIG. 3A. Of course, while a liquid sample is shown being deposited in FIG. 3B, in other embodiments a solid sample may be deposited in a blister chamber via a sample port, as the present disclosure is not so limited.

From the state shown in FIG. 3B, the sample may be allowed to react with the amplification reagents for a predetermined amount of time. In some embodiments, the first blister chamber 1202 may be heated for a predetermined period of time (e.g., with an external heater). Once the solution inside of the first blister chamber 1202 has had a predetermined time to react, an external force may be applied to the first blister chamber 1202. As shown in FIG. 3C, when a threshold force is applied to the first blister chamber, the breakable seal 1206 may be broken and the solution inside of the first blister chamber may be forced into the second blister chamber 1208. That is, the first blister chamber 1202 may collapse under the application of the threshold force, thereby forcing the fluid from the first blister chamber into the second blister chamber 1208. Accordingly, the seal 1206 of the embodiment of FIGS. 3A-3B is a burstable type seal, where fluid from the first blister chamber 1202 is uncontrollably released into the second blister chamber 1208. Once the combined solution of the first blister chamber is moved to the second blister chamber, the combined solution may be given a predetermined time to react. As shown in FIG. 3C, once the combined solution in the second blister chamber 1208 is ready to move to the lateral flow assay strip 1214, the rotary valve 1210 may be rotated (e.g., via a rotary knob or handle) to open the valve as shown by the arrow. Once rotated, the second blister chamber 1208 may be fluidly connected to the third chamber 1212.

As shown in FIG. 3D, once the rotary valve 1210 is opened, the second blister chamber 1208 may be depressed to move the solution contained therein into the third chamber 1212. That is, an external force may be applied to the second blister chamber to collapse the second blister chamber 1208 and move the fluid to the third chamber 1212. Accordingly, the solution is brought into contact with the lateral flow assay strip 1214. In some embodiments, the diagnostic test blister pack 1200 may include a check valve configured to prevent fluid from flowing back to the first blister chamber 1202 from the second blister chamber.

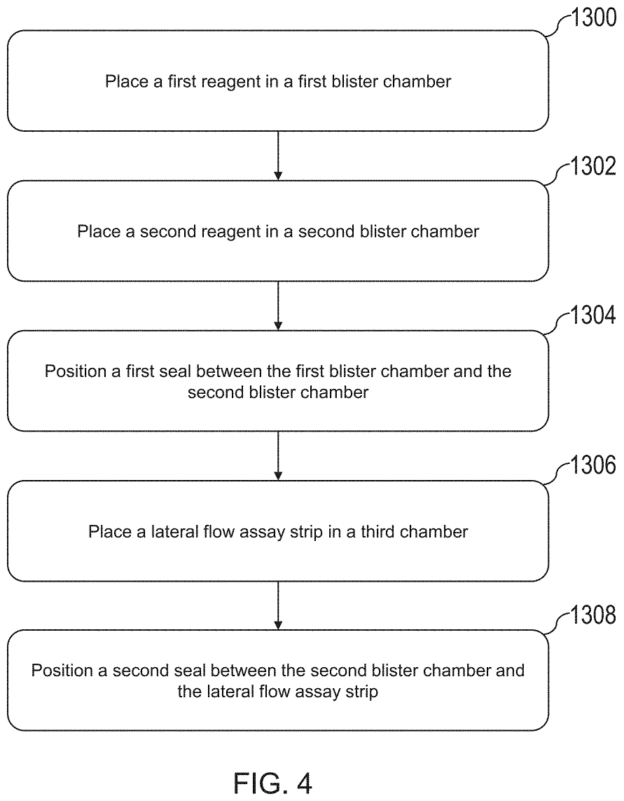

FIG. 4 depicts a flow chart for one embodiment of a method of making a diagnostic test including one or more blister chambers. In step 1300, a first reagent is placed in a first blister chamber. In some embodiments, the first reagent may be a lyophilized solid. In other embodiments, the first reagent may be a liquid solution. In step 1302, a second reagent is placed in a second blister chamber. In some embodiments, the second blister chamber is adjacent to the first blister chamber. The second reagent may be a lyophilized solid or may be a liquid solution. In step 1304, a first seal may be positioned between the first blister chamber and the second blister chamber. In some embodiments, the seal may be a frangible seal configured to release fluid when opened in an uncontrolled manner. In other embodiments, the seal may be a valve configured to release fluid when opened in a controlled manner. In step 1306, a lateral flow assay strip is placed in a third chamber. In step 1308, a second seal is positioned between the second blister chamber and the third chamber (i.e., between the second chamber and the lateral flow assay strip). Accordingly, the diagnostic test made by the method of FIG. 4 may include three chambers arranged in sequence. That is, the first chamber may not be directly connected to the third chamber, but rather indirectly through the second chamber.

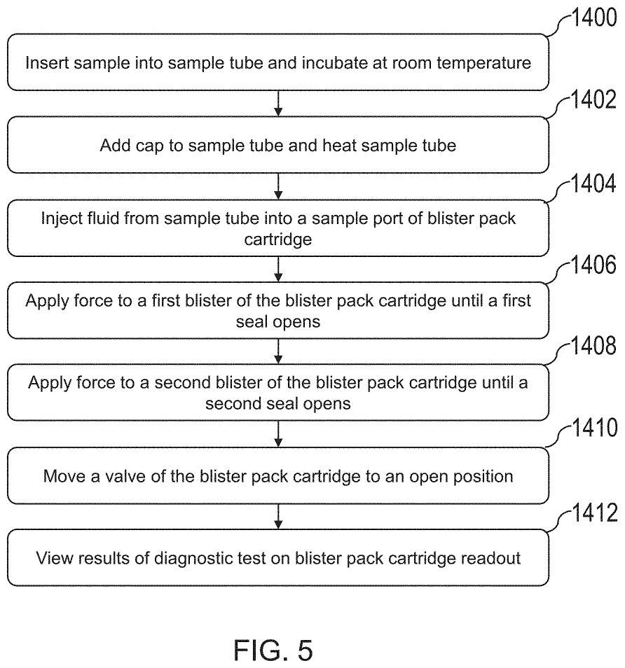

FIG. 5 is a flow chart for one embodiment of performing a diagnostic test. In step 1400, a sample is inserted into a sample tube and incubated at room temperature for a first predetermined period of time. In some embodiments, the sample is taken using a swab (e.g., a nasal swab, cheek swab, etc.). In some embodiments, the sample tube may contain a buffer. In some embodiments, the first predetermined period of time is 10 minutes. In step 1402, a cap is added to the sample tube and the sample tube is heated for a second predetermined period of time. In some embodiments, the cap may contain a lysis mixture which is added to the sample tube. In some such embodiments, the cap may contain a blister containing the lysis mixture which may be depressed and broken by a user to deposit the lysis mixture into the sample tube. In other embodiments, adding the cap may automatically dispense the lysis mixture into the buffer and sample. In still other embodiments, a user may add the lysis mixture to the sample tube (e.g., via a pipette) prior to attaching the cap to the sample tube. In some embodiments, the sample tube may be heated at 95° C., but other temperatures are also envisioned. In some embodiments, the second predetermined period of time is approximately 3 minutes. In some embodiments, the heating is accomplished with boiling water or a fixed heat source. In some embodiments, after heating the mixture in the sample tube may be allowed to cool for 1 minute, though other time periods are also envisioned.

In step 1404 of the process shown in FIG. 5, the fluid from the sample tube is injected into a sample port of a blister pack detection component (e.g., a cartridge). In some embodiments, the mixture may be transferred from the sample tube to the blister pack using a pipette. In some embodiments, the sample port may then be sealed with seal tape, and then shaken (e.g., agitated) for 10 seconds, though other durations are contemplated. In some embodiments, the blister pack may be heated for a third time period. In some embodiments, the third time period is approximately 20 minutes, though other time periods are contemplated. In some embodiments, the cartridge is placed in clothing pocket (e.g., back pocket of pants, front pocket of pants, front pocket of shirt) to heat the cartridge.

In step 1406 of the process shown in FIG. 5, force is applied to a first blister of the blister pack to open a first seal. In some embodiments, the first seal may be a frangible seal configured to burst when the user applies force to the first blister. The first blister may contain a lyophilized amplification mix which is released to mix with the sample when the first seal is opened. In step 1408, force is applied to a second blister of the blister pack until a second seal opens. In some embodiments, the second seal may be a frangible seal configured to burst when the user applies force to the second blister. The second blister may contain a dilution buffer which is released to mix with the sample mixture when the second seal is opened. Once the second seal is opened, the sample mixture may be allowed to mix for a fourth predetermined period of time. In step 1410, a valve of the blister pack is moved to an open position. In some embodiments, moving the valve to the open position may allow the sample mixture to flow to a lateral flow assay strip disposed in the cartridge. Once the valve is opened, the lateral flow assay strip may run automatically. In step 1412, results of the test may be viewed on a readout of the blister pack. In some embodiments, a user may wait 5 minutes for test results to appear on the readout. In some embodiments, the results on the lateral flow strip are interpreted using a mobile software-based application, downloadable to a smart device, such as that described herein.

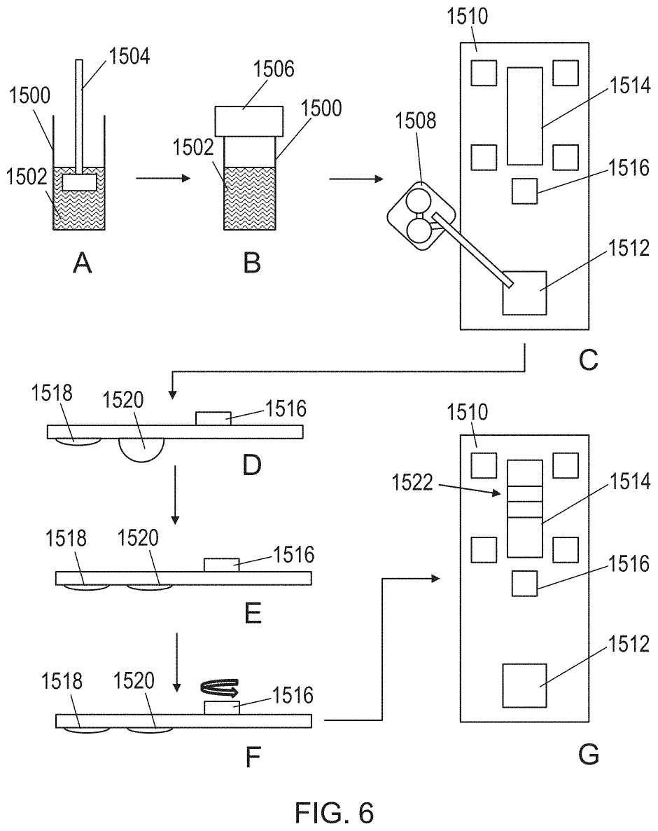

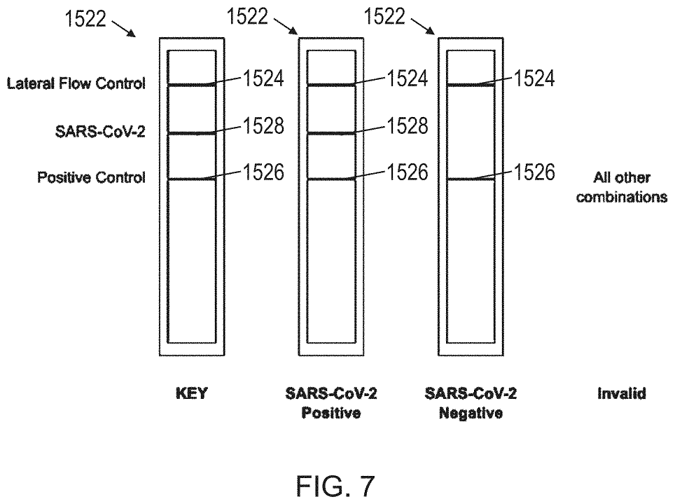

FIG. 6 depicts one embodiment of a diagnostic testing process employing a blister back according to exemplary embodiments described herein. As shown in FIG. 6, a diagnostic testing process may begin at step A with depositing a sample into a sample tube 1500. In particular, the sample may be deposited from a swab 1504 into a buffer solution 1502. In step B, the sample tube 1500 may be sealed with a cap 1506. In some embodiments, the cap may include a lysis mixture which is released into the sample tube. The sealed sample tube may be heated to a predetermined temperature greater than room temperature for a predetermined period of time. Following heating, in step C the sample mixture from the sample tube may be deposited into a blister pack 1510. In particular, a pipette 1508 may be used to inject the sample mixture into a sample port 1512. The view shown in step C is a top view of the blister pack. As shown in FIG. 6, the blister pack also includes a lateral flow assay strip readout 1514 and a valve 1516. Step D depicts a side view of the blister pack. In step D, a first blister 1518 has been depressed by a user. Depressing the first blister may release a lyophilized amplification mix that mixes with the sample mixture. In step E, a second blister 1520 is depressed by a user. Depressing the second blister may release a diluent to mix with the sample mixture. The diluent mixture may ensure that lines displayed on the lateral flow assay readout are clearly defined for ease of interpretation by user or digital device. In step F, the valve 1516 is moved to an open position. Opening the valve 1516 may allow the sample mixture to flow to the lateral flow assay strip disposed in the blister pack. In step G, the readout 1514 may be viewed by a user to determine the result of the test. In particular, lines 1522 appear which provide the test result. Exemplary lines for test results are shown and described further with reference to FIG. 7.

FIG. 7 depicts one embodiment of a lateral flow assay strip readout for SARS-CoV-2 (e.g., Covid-19). As shown in FIG. 7, the readout is configured with three lines 1522. The readout includes a lateral flow control line 1524, a test line 1528 (e.g., for SARS-CoV-2), and a positive control line 1526. For a positive test, all three lines need to be present according to the embodiment of FIG. 7. For a negative test, the lateral flow control line 1524 and positive control line 1526 need to be present, with an absence of the test line 1528. Any other combination of lines indicates an invalid test according to the embodiment of FIG. 7. Of course, any suitable number of lines may be employed for a lateral flow assay strip readout, as the present disclosure is not so limited.

It should be noted that while in some embodiments a detection component of a diagnostic test may be formed as a blister pack including multiple blisters, in other embodiments detection component may have any suitable form including one or more blisters. In some cases, one or more blisters may be employed in any portion of a diagnostic test to facilitate release and combination of different fluid volumes. For example, in some embodiments a blister may be integrated into a cap of a sample tube and may be configured to release lyophilized reagents for amplifying a sample. As another example, in some embodiments a blister pack may be employed to release a diluent to rehydrate one or more reagents inside of a cartridge. Thus, any suitable arrangement employing one or more blisters as described herein may be employed, as the present disclosure is not so limited.

### Blister Pack Diagnostic Test Applications

Diagnostic devices, systems, and methods described herein may be safely and easily operated or conducted by untrained individuals. Unlike prior art diagnostic tests, some embodiments described herein may not require knowledge of even basic laboratory techniques (e.g., pipetting). Similarly, some embodiments described herein may not require expensive laboratory equipment (e.g., thermocyclers). In some embodiments, reagents are contained within a reaction tube and/or a blister pack, such that users are not exposed to any potentially harmful chemicals.

Diagnostic devices, systems, and methods described herein are also highly sensitive and accurate. In some embodiments, the diagnostic devices, systems, and methods are configured to detect one or more target nucleic acid sequences using nucleic acid amplification (e.g., an isothermal nucleic acid amplification method). Through nucleic acid amplification, the diagnostic devices, systems, and methods are able to accurately detect the presence of extremely small amounts of a target nucleic acid. In certain cases, for example, the diagnostic devices, systems, and methods can detect 1 pM or less, or 10 aM or less.

As a result, the diagnostic devices, systems, and methods described herein may be useful in a wide variety of contexts. For example, in some cases, the diagnostic devices and systems may be available over the counter for use by consumers. In such cases, untrained consumers may be able to self-administer the diagnostic test (or administer the test to friends and family members) in their own homes (or any other location of their choosing). In some cases, the diagnostic devices, systems, or methods may be operated or performed by employees or volunteers of an organization (e.g., a school, a medical office, a business). For example, a school (e.g., an elementary school, a high school, a university) may test its students, teachers, and/or administrators, a medical office (e.g., a doctor's office, a dentist's office) may test its patients, or a business may test its employees for a particular disease. In each case, the diagnostic devices, systems, or methods may be operated or performed by the test subjects (e.g., students, teachers, patients, employees) or by designated individuals (e.g., a school nurse, a teacher, a school administrator, a receptionist).

In some embodiments, diagnostic devices described herein are relatively small. In certain cases, for example, a blister pack may be approximately the size of a pen or a marker. Thus, unlike diagnostic tests that require bulky equipment, diagnostic devices and systems described herein may be easily transported and/or easily stored in homes and businesses. In some embodiments, the diagnostic devices and systems are relatively inexpensive. Since no expensive laboratory equipment (e.g., a thermocycler) is required, diagnostic devices, systems, and methods described herein may be more cost effective than known diagnostic tests.

In some embodiments, any reagents contained within a diagnostic device or system described herein may be thermostabilized, and the diagnostic device or system may be shelf stable for a relatively long period of time. In certain embodiments, for example, a blister pack may be stored at room temperature (e.g., 20° C. to 25° C.) for a relatively long period of time (e.g., at least 1 month, at least 3 months, at least 6 months, at least 9 months, at least 1 year, at least 5 years, at least 10 years). In certain embodiments, the blister pack may be stored across a range of temperatures (e.g., 0° C. to 20° C., 0° C. to 37° C., 0° C. to 60° C., 0° C. to 90° C., 20° C. to 37° C., 20° C. to 60° C., 20° C. to 90° C., 37° C. to 60° C., 37° C. to 90° C., 60° C. to 90° C.) for a relatively long period of time (e.g., at least 1 month, at least 3 months, at least 6 months, at least 9 months, at least 1 year, at least 5 years, at least 10 years).

### Target Nucleic Acid Sequences

The diagnostic devices, systems, and methods described herein may be used to detect the presence or absence of any target nucleic acid sequence (e.g., from any pathogen of interest) or multiple target nucleic acid sequences. Target nucleic acid sequences may be associated with a variety of diseases or disorders. In some embodiments, the diagnostic devices, systems, and methods are used to diagnose at least one disease or disorder caused by a pathogen. In certain instances, the diagnostic devices, systems, and methods are configured to detect a nucleic acid encoding a protein (e.g., a nucleocapsid protein) of SARS-CoV-2, which is the virus that causes COVID-19. In some embodiments, the diagnostic devices, systems, and methods are used to diagnose at least one disease or disorder caused by a virus, bacteria, fungus, protozoan, parasite, and/or cancer cell. Of course, a diagnostic test according to exemplary embodiments described herein (e.g., a blister pack) may be employed to detect any desired target nucleic acid sequence, as the present disclosure is not so limited.

### Diagnostic Systems

According to some embodiments, diagnostic systems comprise a sample-collecting component (e.g., a swab) and a diagnostic device. In certain cases, the diagnostic device comprises a blister pack. In some cases, the diagnostic device comprises a detection component (e.g., a lateral flow assay strip). In certain embodiments, the diagnostic device further comprises one or more reagents (e.g., lysis reagents, nucleic acid amplification reagents, CRISPR/Cas detection reagents). Each of the one or more reagents may be in liquid form (e.g., in solution) or in solid form (e.g., lyophilized, dried, crystallized, air jetted). The diagnostic device may also comprise an integrated heater, or the diagnostic system may comprise a separate heater configured to heat one or more chambers or reservoirs of a blister pack or other portion of a diagnostic system. In some embodiments, a heater may be a printed circuit board (PCB) heater that may be integrated into a blister pack.

### Sample Collection

In some embodiments, a diagnostic method comprises collecting a sample from a subject (e.g., a human subject, an animal subject). In some embodiments, a diagnostic system comprises a sample-collecting component configured to collect a sample from a subject (e.g., a human subject, an animal subject). Exemplary samples include bodily fluids (e.g. mucus, saliva, blood, serum, plasma, amniotic fluid, sputum, urine, cerebrospinal fluid, lymph, tear fluid, feces, or gastric fluid), cell scrapings (e.g., a scraping from the mouth or interior cheek), exhaled breath particles, tissue extracts, culture media (e.g., a liquid in which a cell, such as a pathogen cell, has been grown), environmental samples, agricultural products or other foodstuffs, and their extracts. In some embodiments, the sample comprises a nasal secretion. In certain instances, for example, the sample is an anterior nares specimen. An anterior nares specimen may be collected from a subject by inserting a swab element of a sample-collecting component into one or both nostrils of the subject for a period of time. In some embodiments, the sample comprises a cell scraping. In certain embodiments, the cell scraping is collected from the mouth or interior cheek. The cell scraping may be collected using a brush or scraping device formulated for this purpose. The sample may be self-collected by the subject or may be collected by another individual (e.g., a family member, a friend, a coworker, a health care professional) using a sample-collecting component described herein.

### Lysis of Sample

In some embodiments, lysis is performed by chemical lysis (e.g., exposing a sample to one or more lysis reagents) and/or thermal lysis (e.g., heating a sample). Chemical lysis may be performed by one or more lysis reagents. In some embodiments, the one or more lysis reagents comprise one or more enzymes. In some embodiments, the one or more lysis reagents comprise one or more detergents. In some embodiments, cell lysis is accomplished by applying heat to a sample (thermal lysis). In certain instances, thermal lysis is performed by applying a lysis heating protocol comprising heating the sample at one or more temperatures for one or more time periods using any heater described herein. In some embodiments, a lysis heating protocol comprises heating the sample at a first temperature for a first time period.

### Nucleic Acid Amplification

Following lysis, one or more target nucleic acids (e.g., a nucleic acid of a target pathogen) may be amplified. In some cases, a target pathogen has RNA as its genetic material. In certain instances, for example, a target pathogen is an RNA virus (e.g., a coronavirus, an influenza virus). In some such cases, the target pathogen's RNA may need to be reverse transcribed to DNA prior to amplification. In some embodiments, reverse transcription is performed by exposing lysate to one or more reverse transcription reagents. In certain instances, the one or more reverse transcription reagents comprise a reverse transcriptase, a DNA-dependent polymerase, and/or a ribonuclease (RNase). In some embodiments, DNA may be amplified according to any nucleic acid amplification method known in the art.

### LAMP

In some embodiments, the nucleic acid amplification reagents are LAMP reagents. LAMP refers to a method of amplifying a target nucleic acid using at least four primers through the creation of a series of stem-loop structures. Due to its use of multiple primers, LAMP may be highly specific for a target nucleic acid sequence.

### RPA

In some embodiments, the nucleic acid amplification reagents are RPA reagents. RPA generally refers to a method of amplifying a target nucleic acid using a recombinase, a single-stranded DNA binding protein, and a strand-displacing polymerase.

### Nicking Enzyme Amplification Reaction (NEAR)

In some embodiments, amplification of one or more target nucleic acids is accomplished through the use of a nicking enzyme amplification reaction (NEAR) reaction. NEAR generally refers to a method for amplifying a target nucleic acid using a nicking endonuclease and a strand displacing DNA polymerase. In some cases, NEAR may allow for amplification of very small amplicons.

### Molecular Switches

As described herein, a sample undergoes lysis and amplification prior to detection. In certain embodiments, one or more (and, in some cases, all) of the reagents necessary for lysis and/or amplification are present in a single pellet or tablet. In some embodiments, a pellet or tablet may comprise two or more enzymes, and it may be necessary for the enzymes to be activated in a particular order. Therefore, in some embodiments, the enzyme tablet further comprises one or more molecular switches. Molecular switches, as described herein, are molecules that, in response to certain conditions, reversibly switch between two or more stable states. In some embodiments, the condition that causes the molecular switch to change its configuration is pH, light, temperature, an electric current, microenvironment, or the presence of ions and other ligands. In one embodiment, the condition is heat. In some embodiments, the molecular switches described herein are aptamers. Aptamers generally refer to oligonucleotides or peptides that bind to specific target molecules (e.g., the enzymes described herein). The aptamers, upon exposure to heat or other conditions, may dissociate from the enzymes. With the use of molecular switches, the processes described herein (e.g., lysis, decontamination, reverse transcription, and amplification) may be performed in a single test tube with a single enzymatic tablet.

### Detection

In some embodiments, amplified nucleic acids (i.e., amplicons) may be detected using any suitable methods. In some embodiments, one or more target nucleic acid sequences are detected using a lateral flow assay strip (e.g., disposed in a blister pack). In some embodiments, a fluidic sample (e.g., comprising a particle-amplicon conjugate) is configured to flow through a region of the lateral flow assay strip (e.g., a test pad) comprising one or more test lines. In some embodiments, a first test line comprises a capture reagent (e.g., an immobilized antibody) configured to detect a first target nucleic acid and an opaque marking may appear if the target nucleic acid is present in the fluidic sample. The marking may have any suitable shape or pattern (e.g., one or more straight lines, curved lines, dots, squares, check marks, x marks). In certain embodiments, the lateral flow assay strip comprises one or more additional test lines. In some instances, each test line of the lateral flow assay strip is configured to detect a different target nucleic acid. In certain embodiments, the region (e.g., the test pad) of the lateral flow assay strip generating an opaque marking further comprises one or more control lines to indicate that a human (or animal) sample was successfully collected, nucleic acids from the sample were amplified, and that amplicons were transported through the lateral flow assay strip.

### Instructions & Software

In some embodiments, a diagnostic system comprises instructions for using a diagnostic device and/or otherwise performing a diagnostic test method. The instructions may include instructions for the use, assembly, and/or storage of the diagnostic device and any other components associated with the diagnostic system. The instructions may be provided in any form recognizable by one of ordinary skill in the art as a suitable vehicle for containing such instructions. For example, the instructions may be written or published, verbal, audible (e.g., telephonic), digital, optical, visual (e.g., videotape, DVD, etc.) or electronic communications (including Internet or web-based communications). In some embodiments, the instructions are provided as part of a software-based application. In certain cases, the application can be downloaded to a smartphone or device, and then guides a user through steps to use the diagnostic device.

In some embodiments, a software-based application may be connected (e.g., via a wired or wireless connection) to one or more components of a diagnostic system. In certain embodiments, for example, a heater may be controlled by a software-based application. In some cases, a user may select an appropriate heating protocol through the software-based application. In some cases, an appropriate heating protocol may be selected remotely (e.g., not by the immediate user). In some cases, the software-based application may store information (e.g., regarding temperatures used during the processing steps) from the heater.

In some embodiments, a diagnostic system comprises or is associated with software to read and/or analyze test results. In some embodiments, a device (e.g., a camera, a smartphone) is used to generate an image of a test result (e.g., one or more lines detectable on a lateral flow assay strip). In some embodiments, a user may use an electronic device (e.g., a smartphone, a tablet, a camera) to acquire an image of the visible portion of the lateral flow assay strip. In some embodiments, software running on the electronic device may be used to analyze the image (e.g., by comparing any lines or other markings that appear on the lateral flow assay strip with known patterns of markings). That result may be communicated directly to a user or to a medical professional. In some cases, the test result may be further communicated to a remote database server. In some embodiments, the remote database server stores test results as well as user information such as at least one of name, social security number, date of birth, address, phone number, email address, medical history, and medications.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

What is claimed is:

1. A diagnostic test comprising:
a housing;
a first blister chamber formed in the housing containing a first reagent; and
a lateral flow assay strip disposed in the housing.

2. The diagnostic test of claim 1, further comprising:
a fluidic channel between the first blister chamber and the lateral flow assay strip; and
a seal positioned between the first blister chamber and the lateral flow assay strip, wherein applying a threshold force to the first blister chamber is configured to open the seal to fluidly connect the first blister chamber to the fluidic channel.

3. The diagnostic test of claim 2, further comprising:
a second blister chamber formed in the housing containing a second reagent, wherein the second blister chamber is positioned between the seal and the lateral flow assay strip; and
a second seal positioned between the second blister chamber and the lateral flow assay strip.

4. The diagnostic test of claim 3, wherein the second seal is a frangible seal configured to be broken when a second threshold force is applied to the second blister chamber.

5. The diagnostic test of claim 3, wherein the second seal is a valve configured to be moved between a closed state and an open state, wherein in the open state the second blister chamber is fluidly connected to the fluidic channel.

6. The diagnostic test of claim 5, wherein the valve is a rotary valve.

7. The diagnostic test of claim 3, further comprising a third blister chamber containing the lateral flow assay strip.

8. The diagnostic test of claim 7, further comprising a fourth blister chamber containing a third reagent and a third seal positioned between the fourth blister chamber and the fluidic channel.

9. The diagnostic test of claim 8, wherein the fourth blister chamber includes a sample port configured to receive a sample.

10. The diagnostic test of claim 8, wherein the fourth blister chamber is fluidly connected to the first blister chamber and the third seal is positioned between the fourth blister chamber and the first blister chamber, and wherein the first blister chamber is positioned between the fourth blister chamber and the second blister chamber, and wherein the second blister chamber is positioned between the first blister chamber and the third blister chamber.

11. The diagnostic test of claim 8, further comprising a fifth blister chamber containing a fourth reagent and a fourth seal positioned between the fourth blister chamber and the fluidic channel.

12. A method of performing a diagnostic test, comprising:
depositing a sample into a first blister chamber through a sample port formed in a housing;
allowing the sample to react with a first reagent in the first blister chamber to form a first solution;
applying a threshold force to the first blister chamber to break a first frangible seal holding the first solution in the first blister chamber; and
allowing the first solution to flow toward a lateral flow assay strip disposed in the housing.

13. The method of claim 12, wherein allowing the sample to react with the first reagent includes shaking or agitating the housing.

14. The method of claim 12, wherein allowing the sample to react with the first reagent includes heating the first blister chamber with a heater.

15. The method of claim 12, further comprising allowing the first solution to flow to a second blister chamber prior to flowing toward the lateral flow assay strip.

16. The method of claim 15, wherein the second blister chamber contains a second reagent.

17. The method of claim 15, further comprising applying a second threshold force to the second blister chamber to break a second frangible seal between the second blister chamber and the lateral flow assay strip.

18. The method of claim 15, further comprising opening a valve positioned between the second blister chamber and the lateral flow assay strip.

19. A method of making a diagnostic test, comprising:
placing a first reagent in a first blister chamber;
placing a second reagent in a second blister chamber;
positioning a first seal between the first blister chamber and the second blister chamber; and
placing a lateral flow assay strip in a third chamber.

20. The method of claim 19, further comprising positioning a second seal between the second blister chamber and the lateral flow assay strip.
